(19) United States
(12) Patent Application Publication
(10) Pub. No.: US 2007/0167645 A1
(43) Pub. Date: Jul. 19, 2007

(54) Radiation-cured substances

(76) Inventors: Matthias Fies, Krefeld (DE); Holger Endres, Neuss (DE); Jan-Mirco Stachowiak, Essen (DE); Ronald Klagge, Erkrath (DE)

Correspondence Address: COGNIS CORPORATION;PATENT DEPARTMENT, 300 BROOKSIDE AVENUE, AMBLER, PA, 19002, US

(21) Appl. No.: 10/553,483

(22) PCT Filed: Apr. 8, 2004
(86) PCT No.: PCT/EP04/03785

§ 371 Date: Nov. 14, 2006

(30) Foreign Application Priority Data
Apr. 17, 2003 (DE) 103 17 646.2

(51) Int. Cl. C07C 69/52

(52) U.S. Cl. 560/224

(57) ABSTRACT

The disclosed invention relates to mixtures of acrylic or methacrylic compounds containing 1 to 35% by weight and, more particularly, 5 to 25% by weight epoxy (meth)acrylates, based on the total quantity of acrylic or methacrylic compounds, the mixture being obtainable by a process comprising the following steps: a) esterifying hydroxyl compounds with acrylic acid and/or methacrylic acid, b) optionally adding more acrylic acid and/or methacrylic acid and c) reacting the excess acrylic acid and/or methacrylic acid with epoxides in the presence of the esterification product of a). Said mixtures are suitable for use as radiation-curable compositions for coating systems, such as paints, coating compositions and the like.

### FIELD OF THE INVENTION

This invention relates to mixtures of acrylic or methacrylic compounds containing 1 to 35% by weight and, more particularly, 5 to 25% by weight epoxy (meth)acrylates, based on the total quantity of acrylic or methacrylic compounds, the mixture being obtainable by a process comprising the following steps: a) esterifying hydroxyl compounds with acrylic acid and/or methacrylic acid, b) optionally adding more acrylic acid and/or methacrylic acid and c) reacting the excess acrylic acid and/or methacrylic acid with epoxides in the presence of the esterification product of a). The present invention also relates to a process for the production of the mixtures mentioned and to the use of the mixtures as radiation-curable compositions.

### PRIOR ART

EP-B-279303 discloses radiation-curable acrylates obtainable by simultaneous reaction of a) 1 equivalent of a 2- to 6-functional alkoxylated alcohol with b) 0.05 to 1 equivalent of a 2- to 4-basic carboxylic acid or carboxylic acid anhydride and c) 0.1 to 1.5 equivalents of acrylic acid and/or methacrylic acid and reaction of the excess carboxyl groups with an equivalent quantity of an epoxy compound.

EP-A-921168 discloses mixtures of acrylic or methacrylic compounds containing 40 to 95% by weight epoxy (meth)acrylates, based on the total quantity of acrylic or methacrylic compounds, characterized in that the mixtures are obtainable by a process comprising the following successive steps: a) esterifying hydroxyl compounds with acrylic acid or methacrylic acid, b) optionally adding more acrylic acid or methacrylic acid and c) reacting the excess acrylic acid or methacrylic acid with epoxides in the presence of the esterification product of a).

### DESCRIPTION OF THE INVENTION

The problem addressed by the present invention was to provide radiation-curable compositions based on a mixture of acrylic or methacrylic compounds. These compositions would be stable in storage and easy to handle (by virtue in particular of a low viscosity and pourability at 20° C.) and would give coatings combining high hardness with good elasticity. In addition, the composition would be compatible with flatting agents liquid at 20° C. Another problem addressed by the invention was to provide a process for the production of the compositions.

The present invention relates firstly to mixtures of acrylic or methacrylic compounds containing 1 to 35% by weight epoxy (meth)acrylates, based on the total quantity of acrylic or methacrylic compounds, the mixture being obtainable by a process comprising the following steps to be carried out consecutively: a) esterifying hydroxyl compounds (I) with acrylic acid and/or methacrylic acid, b) optionally adding more acrylic acid and/or methacrylic acid and c) reacting the excess acrylic acid and/or methacrylic acid with epoxides in the presence of the esterification product from step a).

In a preferred embodiment, the quantity of the epoxy (meth)acrylates present in the mixture is 5 to 25% by weight and, in a particularly preferred embodiment, of the order of 20% by weight.

Step a)

The compounds (I) to be used in the esterification step a) are hydroxyl compounds. These compounds contain by definition at least one and preferably two or more OH groups per molecule. In a preferred embodiment, reaction products of polyols with α,ω-dicarboxylic acids are used as the hydroxyl compounds (I) in step a). αω-dicarboxylic acids containing a total of 2 to 10 carbon atoms are preferred, adipic acid being particularly preferred. Particularly suitable phenols are, for example, glycerol and trimethylol propane and, more particularly, products of the addition of 1 to 10 mol ethylene oxide onto glycerol and/or trimethylol propane. A reaction product of 2 mol of an addition product of TMP-3EO and 1 mol adipic acid (TMP-3EO being an addition product of 3 mol ethylene oxide onto 1 mol trimethylol propane) is most particularly preferred as the hydroxyl compound (I).

The esterification step a), i.e. the reaction of hydroxyl compounds (I) and acrylic acid and/or methacrylic acid, may be carried out in the presence of a catalyst.

The esterification step a) is preferably carried out in the presence of a polymerization inhibitor, such as hydroquinone monomethyl ether for example. In addition, the esterification step a) is preferably carried out in the presence of a decoloring agent, such as hypophosphorous acid.

The esterification step a) is carried out by measuring the quantity of acrylic and/or methacrylic acid used so that it is sufficient to esterify all the free OH groups of the hydroxyl compound (I). In a preferred embodiment, the acrylic and/or methacrylic acid is used in excess, so that, on the one hand, all the OH groups of the hydroxyl compound (I) are present in esterified form after the esterification reaction and, on the other hand, excess acrylic and/or methacrylic acid is still present. In this variant, the optional step b) of adding more acrylic and/or methacrylic acid may be omitted.

The esterification step a) may be carried out in the absence of a solvent or in a solvent. The esterification may be carried out under azeotropic conditions at elevated temperature, generally at 40 to 120° C. and preferably at 70 to 115° C., hydrocarbons, such as aliphatic, cycloaliphatic or aromatic hydrocarbons or mixtures thereof, preferably those with a boiling range of 70 to 120° C., having proved to be particularly suitable as entraining agents for removing the water of reaction. Suitable aliphatic hydrocarbons are, for example, hexane and its isomers and hydrocarbon mixtures with a boiling range of 60 to 95° C. Particularly preferred entraining agents are cyclohexane, methyl cyclohexane and toluene. The quantity of hydrocarbon added is not critical. Depending on the apparatus used, the quantity by weight added may be between 0.1 and 2 times the quantity of reaction mixture of (meth)acrylic acid and hydroxyl compound (I). A ratio of reaction mixture to hydrocarbon of 1:0.20 to 1:0.8 is particularly advantageous.

The water of reaction formed in the esterification step b) is preferably removed azeotropically.

After the esterification step a), the solvent, for example the hydrocarbon, may be removed from the reaction mixture by distillation, optionally under reduced pressure. If desired, the esterification catalyst may be suitably neutralized, for example by addition of tertiary amines or alkali metal hydroxides.

Step b)

As already mentioned, step b) is optional. Whether, and how much, acrylic or methacrylic acid is added in step b) depends on the quantity of acrylic or methacrylic acid used in step a) and the quantity of epoxy (meth)acrylate required in the end product, i.e. on completion of step c).

Step c)

Step c) is a reaction of the excess acrylic acid and/or methacrylic acid with epoxides in the presence of the esterification product from step a). The reaction is conducted in such a way that the acrylic and/or methacrylic acid present in almost completely consumed. The quantity of epoxides to be used may readily be calculated from the acid value of the mixture of esterification product from step a) and the acrylic and/or methacrylic acid present, as well known to the expert.

Step c) is preferably carried out in the presence of a catalyst. Suitable neutralization catalysts are, in particular, acidic or basic compounds and compounds containing tin or chromium. Preferred acidic catalysts are the oxo acids of the elements of the 6^{th} Main Group of the Periodic System and organic derivatives thereof. Sulfuric acid and p-toluenesulfonic acid are particularly preferred. Preferred basic catalysts are nitrogen- or phosphorus-containing compounds. Tertiary amines, such as for example triethylamine, tributylamine and triethanolamine, and phosphines, such as triphenyl phosphine for example, are particularly preferred. In a particularly preferred embodiment of the invention, the catalyst used in step c) is the same as that used in the esterification step a). The catalyst is used in particular in a quantity of 0.1 to 10% by weight, preferably 0.3 to 5% by weight and more preferably 0.5 to 2% by weight, based on the mixture as a whole used in step c).

The epoxides used in step c) are compounds containing 1 to 5, preferably 1 to 3 and more particularly 2 epoxide groups per molecule. The molecular weight of such compounds is preferably below 2,000 g/mol, more preferably below 1,100 g/mol and most preferably below 500 g/mol. Epoxides containing an aromatic group, preferably a diphenyl group, are particularly suitable.

Suitable epoxides are, for example, epoxidized olefins, glycidyl esters of saturated or unsaturated carboxylic acids or glycidyl ethers of aliphatic or aromatic polyols. Products such as these are commercially available in large numbers. Polyglycidyl compounds of bisphenol A and glycidyl ethers of polyfunctional alcohols, for example butanediol, glycerol and pentaerythritol, are particularly preferred.

Diepoxides, i.e. compounds containing two oxirane rings (epoxy groups) per molecule, are particularly preferred. The following are examples of such compounds:
4-vinyl-2-cyclohexene diepoxide
1,4-cyclohexane dimethanol diglycidyl ether
1,4-butane diol diglycidyl ether, neopentyl glycol diglycidyl ether
3-(bis-(glycidyloxymethyl)-methoxy)-propane-1,2-diol
diglycidyl-1,2,3,6-tetrahydrophthalate
3,4-epoxycyclohexylmethyl-3,4-epoxycyclohexane carboxylate
3,4-epoxy-6-methylcyclohexylmethyl-3,4,6-methyl cyclohexane carboxylate
diglycidyl-1,2-cyclohexane dicarboxylate
bis-(3,4-epoxycyclohexylmethyl)-adipate
2-(3,4-epoxycyclohexyl)-5,5-spiro-3,4-epoxy)-cyclohexane meta-dioxane
bisphenol A diglycidyl ether
bisphenol F diglycidyl ether
bisphenol H diglycidyl ether

The diglycidyl ethers based on bisphenol A and bisphenol F are particularly preferred.

If desired, an inhibitor may be added to the end product. This may be done either before or after step c).

Process

The present invention also relates to a process for the production of mixtures of acrylic or methacrylic compounds containing 1 to 35% by weight epoxy (meth)acrylates, based on the total quantity of acrylic or methacrylic compounds, the mixture being obtainable by carrying out the following steps consecutively: a) esterifying hydroxyl compounds (I) with acrylic acid and/or methacrylic acid, b) optionally adding more acrylic acid and/or methacrylic acid and c) reacting the excess acrylic acid and/or methacrylic acid with epoxides in the presence of the esterification product from step a).

The individual steps or parameters of the process and preferred embodiments are as already described in the foregoing.

Use

The present invention also relates to the use of the mixtures obtainable as described above as radiation-curable compositions for coating systems, such as paints, coating compositions and the like.

In another embodiment, one or more dimerdiol (meth)acrylates with a degree of esterification of at least 50% is/are added to the above-described mixtures produced in accordance with the invention in quantities of 0.01 to 20% by weight, preferably 2 to 15% by weight and, more particularly, 3 to 10% by weight, based on the mixtures as a whole, for their use as radiation-curable compositions for coating systems. The coating systems produced using these modified mixtures are distinguished by excellent flatting effects.

The technical concept of flatting as applied to coating systems is discussed in the following. Certain applications require coating compositions which give gloss-reduced surfaces after application and curing. In the prior art, this goal is achieved by the use of substances which, in paint systems, reduce the surface gloss of the cured paint to a more or less considerable extent. Such substances are known to the expert as flatting agents. Accordingly, flatting agents are substances used as additives to reduce the surface gloss of cured paints or coatings. The term “flatting agent” is used in its universally accepted sense in the present specification.

The dimerdiol (meth)acrylates mentioned represent flatting agents which are liquid at 20° C. In terms of handling behavior and incorporation in coating systems, these dimerdiol (meth)acrylates represent a major advantage over conventional flatting agents which are solid at 20° C. In particular, dimerdiol (meth)acrylates do not increase the viscosity of the coating systems to which they are added. In addition, even relatively thick paint coatings can readily be flatted with dimerdiol (meth)acrylates.

Dimerdiol (meth)acrylates in the context of the invention are esters of dimerdiol and acrylic and/or methacrylic acid. The dimer diol on which the dimerdiol (meth)acrylates are based may be present as such or in technical quality. As already mentioned, the dimerdiol (meth)acrylates to be used in accordance with the invention have a degree of esterification of at least 50%. Their degree of esterification is preferably at least 80% and, more particularly, at least 92%. In a most particularly preferred embodiment, the degree of esterification is 100%, i.e. the esters are full esters, i.e. dimerdiol di(meth)acrylates. In the interests of clarity, it is pointed out that a degree of esterification of x % means that, where 1 mol dimer diol is used to produce a dimerdiol (meth)acrylate, x % of all the OH groups present therein are esterified.

Dimerdiol (meth)acrylates can be obtained, for example, by esterification of dimer diols with acrylic acid or methacrylic acid or mixtures of acrylic and methacrylic acid. The dimer diols may be used either as such or in technical quality (see below).

Dimerdiols are well-known commercially available compounds which are obtained, for example, by reduction of dimer fatty acid esters. The dimer fatty acids on which these dimer fatty acid esters are based are carboxylic acids which may be obtained by oligomerization of unsaturated carboxylic acids, generally fatty acids, such as oleic acid, linoleic acid, erucic acid and the like. The oligomerization is normally carried out at elevated temperature in the presence of a catalyst, for example of clay. The substances obtained—dimer fatty acids of technical quality—are mixtures in which the dimerization products predominate. However, small amounts of higher oligomers, more particularly so-called trimer fatty acids, are also present. Dimer fatty acids are commercially available products and are marketed in various compositions and qualities. Abundant literature is available on the subject of dimer fatty acids, cf. for example the following articles: Fette & Öle 26 (1994), pages 47-51; Speciality Chemicals 1984 (May Number), pages 17, 18, 22-24. Dimerdiols are well-known among experts, cf. for example a more recent article in which inter alia the production, structure and chemistry of the dimerdiols are discussed: Fat Sci. Technol. 95 (1993), No. 3, pages 91-94. According to the invention, dimerdiols preferred as raw materials for the production of dimerdiol (meth)acrylates are those which have a dimer content of at least 70% and more particularly 90% and in which the number of carbon atoms per dimerdiol molecule is mainly in the range from 36 to 44.

Basically, the use of the above-mentioned mixtures of mixtures obtainable by the process according to the invention and the dimerdiol (meth)acrylates for coating systems is not confined to particular substrates. Examples of suitable substrates (i.e. materials to be coated) are, for example, glass, metal, wood, paper, ceramic, plastic. Glass is a particularly preferred substrate.

### EXAMPLES

### Example 1

### Production of a Hydroxyl Compound (Initial Product)

500.0 g (1.81 mol) of an addition product of 3 mol ethylene oxide onto 1 mol trimethylol propane were reacted with 133.0 g (0.91 mol) adipic acid in the presence of 12.2 g methanesulfonic acid.

### Example 2 (Invention)

The hydroxyl compound obtained in Example 1 was reacted with 432 g (6.0 mol) acrylic acid in the presence of the catalyst present in that compound and 12.2 g of a 50% aqueous solution of hypophosphorous acid and 1.2 g (900 ppm) hydroquinone monomethyl ether to form a polyester acrylate. An air stream was passed through the reaction vessel to activate the inhibitor. The reaction took place over 23 hours at 80° C. Water of reaction formed was distilled off during the reaction together with 66.2 g of unreacted acrylic acid. An intermediate product with an acid value of 56 mg KOH/g was obtained. 264.6 g (0.72 mol) bisphenol A diglycidyl ether were added to the reaction mixture containing the intermediate product mentioned and more unreacted acrylic acid and, after the addition of 24.5 g triethylamine (catalyst), the whole was reacted for 7 hours at 90° C.

### Example 3

50.0 g dimerdiol diacrylate were stirred into and uniformly distributed in the product obtained as described in Example 2. The paint thus obtained was applied to a glass plate in a layer thickness of 100 μm and cured with UV light. The coating thus obtained showed an excellent flatting effect.

1-7. (canceled)

8. A composition comprising a mixture of acrylic or methacrylic compounds containing 1 to 35% by weight epoxy (meth)acrylates, based on the total quantity of acrylic or methacrylic compounds, the mixture being obtainable by a process comprising the following steps carried out consecutively:
a) esterifying one or more hydroxyl compounds (I) with acrylic acid and/or methacrylic acid,
b) optionally adding more acrylic acid and/or methacrylic acid, and
c) reacting the excess acrylic acid and/or methacrylic acid with one or more epoxides in the presence of the esterification product from step a).

9. The composition according to claim 8, wherein the hydroxyl compounds (I) in step a) are selected from reaction products of polyols with α,ω-dicarboxylic acids.

10. The composition according to claim 8, wherein the hydroxyl compounds (I) in step a) are selected from reaction products of addition products of 1 to 10 mol ethylene oxide onto glycerol or trimethylol propane or a combination thereof with α,ω-dicarboxylic acids.

11. The composition according to claim 8, wherein the hydroxyl compounds (I) in step a) are selected from reaction products of addition products of 1 to 10 mol ethylene oxide onto glycerol or trimethylol propane or a combination thereof with adipic acid.

12. The composition according to claim 8, wherein the mixture of acrylic or methacrylic compounds contains 5 to 25% by weight of the epoxy (meth)acrylates.

13. The composition according to claim 8, wherein the one or more epoxides are selected from the group comprising polyglycidyl compounds of bisphenol A, glycidyl ethers of polyfinctional alcohols, and diepoxides.

14. A radiation-curable coating composition comprising a composition according to claim 8.

15. A flatting composition comprising
(A) a coating component comprising a mixture of acrylic or methacrylic compounds containing 1to 35% by weight epoxy (meth)acrylates, based on the total quantity of acrylic or methacrylic compounds, the mixture being obtainable by a process comprising the following steps carried out consecutively:
a) esterifying one or more hydroxyl compounds (I) with acrylic acid and/or methacrylic acid,
b) optionally adding more acrylic acid and/or methacrylic acid, and
c) reacting the excess acrylic acid and/or methacrylic acid with one or more epoxides in the presence of the esterification product from step a); and
(B) 0.01 to 20% by weight, based on the weight of the flatting compositions as a whole, of a dimerdiol component comprising one or more dimerdiol (meth)acrylates with a degree of esterification of at least 50%.

16. The flatting composition according to claim 15, wherein the mixture of acrylic or methacrylic compounds in the coating component (A) contains 5 to 25% by weight of the epoxy (meth)acrylates.

17. The flatting composition according to claim 15, wherein, in component (A), the hydroxyl compounds (I) in step a) are selected from reaction products of polyols with α,ω-dicarboxylic acids.

18. The flatting composition according to claim 15, wherein, in component (A), the hydroxyl compounds (I) in step a) are selected from reaction products of addition products of 1 to 10 mol ethylene oxide onto glycerol or trimethylol propane or a combination thereof with α,ω-dicarboxylic acids.

19. The flatting composition according to claim 15, wherein, in component (A), the hydroxyl compounds (I) in step a) are selected from reaction products of addition products of 1 to 10 mol ethylene oxide onto glycerol or trimethylol propane or a combination thereof with adipic acid.

20. The flatting composition according to claim 15, wherein the dimerdiol (meth)acrylates of component (B) have a degree of esterification of at least 80%.

21. The flatting composition according to claim 15, wherein the dimerdiol (meth)acrylates of component (B) have a degree of esterification of at least 92%.

22. The flatting composition according to claim 15, wherein the one or more epoxides of component (A) are selected from the group comprising polyglycidyl compounds of bisphenol A, glycidyl ethers of polyfunctional alcohols, and diepoxides.

23. The flatting composition according to claim 15, wherein the dimerdiol component (B) is present in the amount of 2 to 15% by weight, based on the weight of the flatting composition as a whole.

24. The flatting composition according to claim 15, wherein the dimerdiol component (B) is dimerdiol diacrylate.

25. A glass material coated with the flatting composition according to claim 15.

26. A process for the production of a mixture of acrylic or methacrylic compounds, comprising carrying out the following steps consecutively:
a) esterifying one or more hydroxyl compounds (I) with acrylic acid and/or methacrylic acid,
b) optionally adding more acrylic acid and/or methacrylic acid, and
c) reacting the excess acrylic acid and/or methacrylic acid with one or more epoxides in the presence of the esterification product from step a),
in a manner and with amounts of reactants such that the resulting product contains 1 to 35% by weight of epoxy (meth)acrylates, based on the total quantity of acrylic or methacrylic compounds.
